(19) United States
(12) Patent Application Publication
(10) Pub. No.: US 2025/0367026 A1
(43) Pub. Date: Dec. 4, 2025

(54) HAND WARMER WITH SILICONE OUTER COVER

(71) Applicant: Shenzhen Zheyi Technology co., Ltd., Shenzhen (CN)

(72) Inventor: GUOLONG GAO, Shenzhen (CN)

(21) Appl. No.: 19/188,087

(22) Filed: Apr. 24, 2025

(30) Foreign Application Priority Data
May 31, 2024 (CN) 202421246576.1

(51) Int. Cl. A61F 7/08; A61F 7/00; H05B 1/02

(52) U.S. Cl. CPC A61F 7/08; A61F 7/007; H05B 1/0252; A61F 2007/0036; A61F 2007/0078; A61F 2007/0087

(57) ABSTRACT

The present disclosure discloses a hand warmer, comprising a housing, a battery, a heating control circuit electrically connected to the battery, and a heating element electrically connected to the heating control circuit. The outer surface of the housing is provided with a heat-conducting portion for transferring heat generated by the heating element. The outer surface of the heat-conducting portion is covered with a soft silicone cover. The edge of the soft silicone cover is provided with a hooking portion, wherein an inner wall of the hooking portion clasps onto the housing, and an outer wall of the hooking portion is compression-fixed by the housing to prevent detachment. The soft silicone cover of the present disclosure protects the heat-conducting portion from scratches, enhances tactile comfort during use, and reduces heat dissipation rates to extend the hand warmer operational duration.

### CROSS-REFERENCE TO RELATED APPLICATIONS

This application claims priority to Chinese Patent Application No. 202421246576.1 filed on May 31, 2024, the content of which is hereby incorporated by reference in its entirety.

### TECHNICAL FIELD

The present disclosure relates to the technical field of portable heating devices, and more particularly, to a hand warmer featuring a soft silicone cover optimized for tactile comfort and thermal management.

### BACKGROUND

Current market offerings for hand-warming devices are diverse, including traditional hot water bottles and modern hand warmers utilizing heating elements (e.g., heating plates, heating wires). Due to their compact size, ease of use, and portability, hand warmers have gained widespread adoption in recent years.

Typically, the outer shell of a hand warmer is equipped with a heat-conducting portion (e.g., metal plates such as aluminium sheets) to transfer internally generated heat to the human body. However, direct contact between the user's hand and the metallic heat-conducting portion results in rigid tactile feedback, lacking softness and comfort. Furthermore, existing heat-conducting portions are often exposed to ambient air, accelerating heat dissipation and thereby increasing battery energy consumption while shortening operational duration. Additionally, the unprotected heat-conducting portion is prone to scratches or wear.

Therefore, there is still room for improvement in the user experience and energy efficiency of existing technologies.

### SUMMARY

In view of this, the present disclosure is designed to provide an improved hand warmer to achieve the following objectives: protecting the heat-conducting portion from scratches, enhancing tactile comfort, and extending operational duration by reducing heat dissipation rates.

To realize the above objectives, the present disclosure adopts the following technical solutions:

A hand warmer with silicone cover comprises: a housing, a battery, a heating control circuit electrically connected to the battery, and a heating element electrically connected to the heating control circuit. An outer surface of the housing is provided with a heat-conducting portion for transferring heat generated by the heating element. A soft silicone cover is sleeved over the heat-conducting portion and at least a portion of the housing. The soft silicone cover is configured as a flat pouch-like structure and provided with an outwardly extending hooking portion. The housing is provided with a slot to accommodate the hooking portion, thereby preventing detachment of the soft silicone cover from the housing.

The housing is formed by mating a first housing and a second housing. The edge of the soft silicone cover is provided with a hooking portion, wherein an inner wall of the hooking portion clasps onto the first housing, and an outer wall is compression-fixed by the second housing.

The soft silicone cover is a sheet-like structure that fully covers the heat-conducting portion.

Alternatively, the soft silicone cover is a pouch-like structure enveloping both the housing and the heat-conducting portion.

The soft silicone cover defines a receiving cavity, with both the height and width of the cavity being 0.1-0.13 mm smaller than the height and width of the housing.

The soft silicone cover is provided with an installation opening connected to the receiving cavity.

At the installation opening, the soft silicone cover extends outward to form an L-shaped hooking portion, and the housing is provided with a slot to accommodate the L-shaped hooking portion.

A thickness of the soft silicone cover ranges from 0.8 mm to 1.2 mm.

The outer surface of the soft silicone cover is provided with an anti-slip textured structure to enhance grip stability.

Compared to existing technologies, the present disclosure delivers the following advantages: the soft silicone cover over the heat-conducting portion replaces conventional rigid metallic materials, providing a gentler and more comfortable tactile experience. The warmth transferred through the silicone cover creates a milder heating sensation, significantly improving user comfort.

The soft silicone cover protects the heat-conducting portion, effectively preventing scratches, impacts, or collision damage during use.

With a lower thermal dissipation efficiency than metallic heat-conducting portions, the silicone material isolates direct contact between the heat-conducting portion and ambient air, thereby slowing heat loss and substantially extending battery life.

### BRIEF DESCRIPTION OF DRAWINGS

Many aspects of the embodiment can be better understood with reference to the following drawings. The components in the drawings are not necessarily drawn to scale, the emphasis instead being placed upon clearly illustrating the principles of the embodiment.

FIG. 1 is an isometric view of a hand warmer with silicone outer cover in accordance with an exemplary embodiment of the present disclosure.

FIG. 2 is a schematic isometric view of the soft silicone cover detached from the housing of the hand warmer in FIG. 1.

FIG. 3 is a schematic isometric view of a second housing detached from the housing in FIG. 2.

FIG. 4 is a schematic isometric exploded view of the hand warmer in FIG. 1.

FIG. 5 is a schematic isometric cross-sectional view of the hand warmer in FIG. 1.

FIG. 6 is an enlarged schematic view of part A of the hand warmer in FIG. 5.

FIG. 7 is a schematic isometric view of the hand warmer in FIG. 1 with the housing removed, leaving only the soft silicone cover.

FIG. 8 is a schematic isometric view of a second exemplary embodiment of the hand warmer according to the present disclosure.

FIG. 9 is a schematic isometric view of the hand warmer in FIG. 8 with the housing opened.

FIG. 10 is a schematic isometric exploded view of the hand warmer in FIG. 8.

FIG. 11 is a schematic cross-sectional view of the hand warmer in FIG. 8.

FIG. 12 is an enlarged schematic view of part B of the hand warmer in FIG. 11.

### DESCRIPTION OF EMBODIMENTS

Embodiment of the present disclosure will be described in desecond electrical connector in conjunction with the drawings. It should be noted that the figures are illustrative rather than limiting. The figures are not drawn to scale, do not illustrate every aspect of the described embodiment, and do not limit the scope of the present disclosure.

In the disclosure, it should be noted that directional indicators (e.g., upper, lower, left, right, front, rear, etc.) in the embodiments of the present disclosure are solely used to explain the relative positional relationships, movements, or conditions of components under a specific orientation (as shown in the accompanying drawings). If the orientation changes, these directional indicators shall adjust accordingly.

In the present disclosure, unless explicitly defined or limited otherwise, terms such as “connected,” “fixed,” or similar expressions shall be construed broadly. For example, a “connection” may refer to a fixed connection, a detachable connection, or an integral formation; a mechanical connection or an electrical connection; a direct connection or an indirect connection via an intermediary; or an internal linkage between two components or their interaction. The specific meaning of these terms shall be interpreted by those skilled in the art based on contextual understanding.

Furthermore, descriptions involving terms such as “first,” “second,” etc., are provided for illustrative purposes only and shall not be construed as implying relative importance or implicitly specifying the quantity of technical features. Thus, features prefixed with “first” or “second” may explicitly or implicitly include at least one such feature.

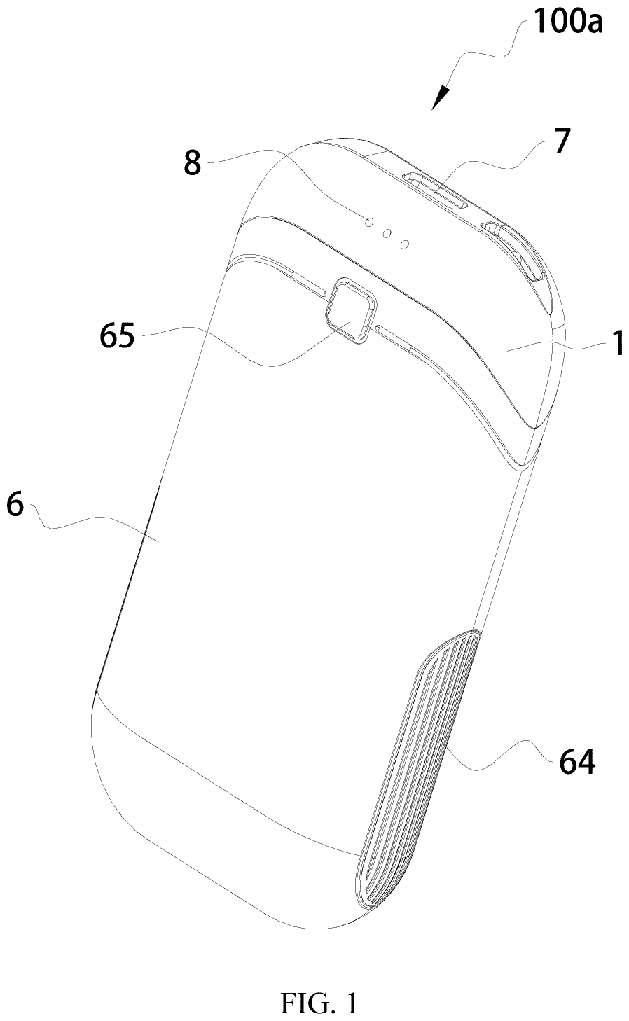

Referring to FIGS. 1 to 7, the present disclosure provides a hand warmer 100a. The hand warmer 100a includes a housing 1, a battery 2, a heating control circuit 3 electrically connected to the battery 2, and a heating element 4 electrically connected to the heating control circuit 2. The outer surface of the housing 1 is provided with a heat-conducting portion 5 for transferring heat generated by the heating element 3. Specifically, in the embodiment, both the battery 2 and the heating control circuit 3 are disposed inside the housing 1. Alternatively, in other embodiments, the battery 2 and heating control circuit 3 may be positioned outside the housing 1. The battery 2 supplies power to electrical components within the hand warmer 100a, while the heating control circuit 3 controls the activation/deactivation status and heating temperature of the heating element 4, as well as manages the charging/discharging process of the battery 2. The heating element 4 converts electrical energy into thermal energy, which is transferred via the heat-conducting portion 5 to human contact areas (e.g., hands or feet). The heating element 4 may adopt common heating components such as a heating plate or heating wire.

As an implementation, the heating control circuit 3 includes a master control unit, a temperature sensor, a charging/discharging circuit, and a power button. The master control unit, in conjunction with the power button, regulates the activation/deactivation of the heating element 4, while the temperature sensor ensures temperature control to prevent overheating or insufficient heating. The charging/discharging circuit manages the battery 2 charging and discharging. Additionally, the heating control circuit 3 in the embodiment includes an external charging interface to power other electronic devices (e.g., smartphones), thereby enabling the hand warmer 100a to function as a portable power bank.

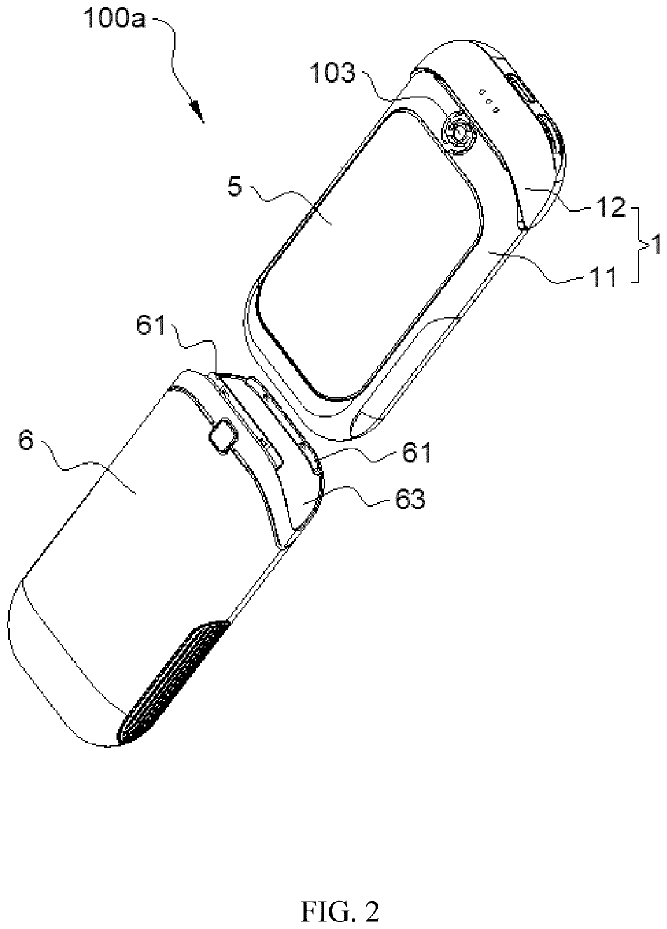

The housing 1 may be fabricated from metal or heat-resistant plastic. Its shape may be a flat rectangular block (as shown in FIGS. 1 and 2), a cylinder, or other ergonomic forms designed for easy grip.

The heat-conducting portion 5 may utilize metals with high thermal conductivity, such as aluminum, copper, iron, or steel. When the housing 1 itself is metallic, the heat-conducting portion 5 may serve as an integral part of the housing 1. Notably, the heat-conducting portion 5 may alternatively be a separately attached metal heat-conducting plate on the outer surface of the housing 1. For example, in the embodiment, the heat-conducting portion 5 is an independently aluminium plate.

To enhance user experience and functionality, the hand warmer 100a of the present disclosure is provided with a soft silicone cover 6 on the outer surface of the heat-conducting portion 4. The soft silicone cover 6 may be fixed to the heat-conducting portion 5 via adhesion, clasping, compression, or envelopment. The soft silicone cover 6 tightly adheres to the heat-conducting portion 5 to ensure efficient heat transfer and avoid thermal efficiency loss due to poor contact.

Referring to FIGS. 1 to 3, in the embodiment, the soft silicone cover 6 is a pouch-like structure that envelopes both the housing 1 and the heat-conducting portion 5. The soft silicone cover 6 acts as a complete outer layer, closely conforming to the outer surfaces of the housing 1 and heat-conducting portion 5 to achieve optimal thermal conduction. During operation, the heat-conducting portion 5 transfers heat from the heating element 4 to the soft silicone cover 6, allowing the user (e.g., via hand contact) to receive warmth through the soft silicone cover 6.

The soft silicone cover 6 may be made of silicone, rubber, PVC, or similar materials. Preferably, the soft silicone cover 6 is fabricated from silicone, which offers superior tactile comfort and heat resistance. By covering the heat-conducting portion 5, the soft silicone cover 6 protects the metallic heat-conducting portion 5 from scratches or abrasion.

Moreover, the soft silicone material provides a plush, comfortable grip compared to the rigid metallic surfaces of conventional designs. The warmth transferred through the soft silicone cover 6 is gentler, significantly improving user comfort.

Additionally, the silicone material's lower thermal conductivity slows heat dissipation relative to the metallic heat-conducting portion 5. By isolating the heat-conducting portion 5 from direct contact with ambient air, the soft silicone cover 6 reduces heat loss. Consequently, the battery 2 of the hand warmer 100a meets user heating demands and thermal loss requirements at lower power outputs, substantially extending its operational duration.

Referring to FIGS. 1 and 2, the heat-conducting portion 5 is disposed on the front surface of the housing 1 or simultaneously on both the front and rear surfaces. In existing technologies, where no soft silicone cover 6 envelops the housing 1 and heat-conducting portion 5, heat concentrates on the front and rear sides of the housing 1 during operation, while the left and right sides exhibit significantly lower temperatures. When users grip the hand warmer, their hands primarily contact the left and right sides of the housing 1, where minimal warmth is perceived. This creates an uneven thermal experience between the front/rear and left/right sides, compromising overall heating effectiveness.

In the embodiment of the present disclosure, the pouch-like soft silicone cover 6 envelops the housing 1, covering its front, rear surfaces, and left/right sidewalls. Due to the soft silicone cover 6 excellent thermal conductivity, the portions adhering to the left/right sides of the housing 1 absorbs heat via conduction, achieving relatively balanced temperatures across all directions. Specifically, the soft silicone cover 6 in the embodiment is made of silicone, which uniformly transfers heat from the housing 1 to all surfaces (front, rear, left, and right), expanding the effective heating area and enhancing user experience. When gripping the hand warmer 100a, users perceive consistent warmth across all contact points with the soft silicone cover 6, experiencing enveloping heat-unlike conventional designs where only the front/rear surfaces are warm.

Furthermore, the soft silicone cover 6 employs thermally conductive silicone material infused with thermal conductive powder. Compared to standard silicone, the material exhibits higher thermal conductivity, enabling faster temperature rise. Even when covering the heat-conducting portion 5, heat rapidly transfers to the outer surface of the soft silicone cover 6, reaching the desired temperature quickly and eliminating prolonged waiting times.

The thermally conductive silicone is molded by blending organic silicone with thermal conductive powder (e.g., metal oxides such as aluminum oxide (Al₂O₃) or magnesium oxide (MgO), or metal nitrides such as aluminum nitride (AlN) or boron nitride (BN)) during manufacturing, significantly enhancing thermal performance.

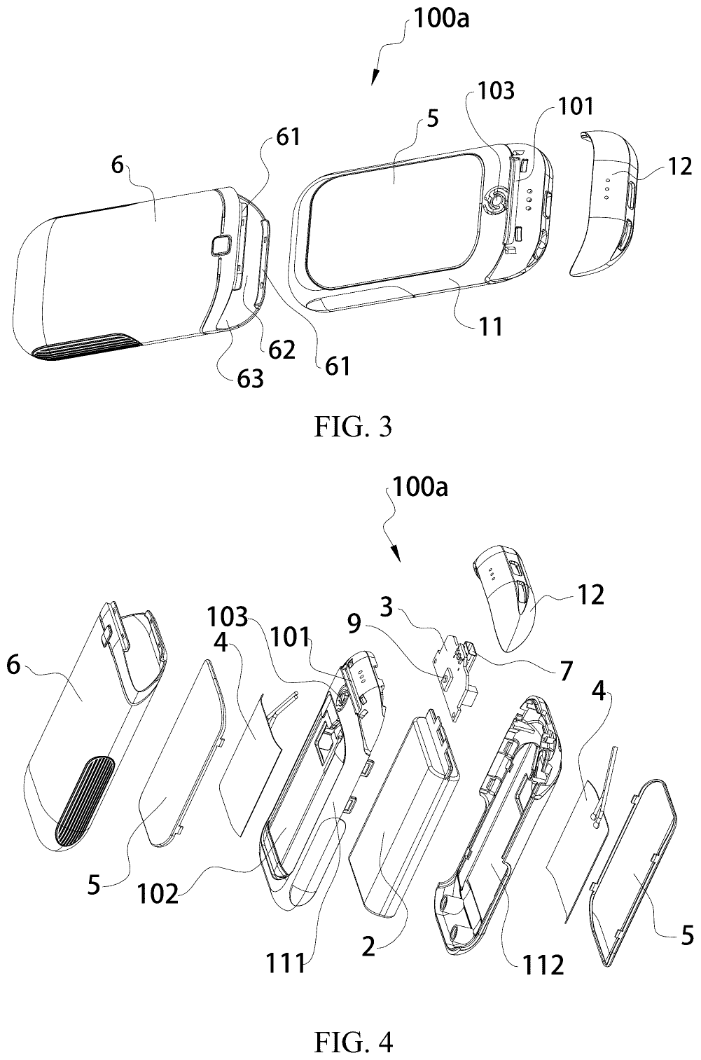

Referring to FIGS. 2 and 3, in the embodiment, the housing 1 is formed by mating a first housing 11 and a second housing 11. The edge of the soft silicone cover 6 is provided with a hooking portion 61, where the inner wall 601 of the hooking portion 61 clasps onto the first housing 11, and the outer wall 602 is compression-fixed by the second housing 12.

In the embodiment, referring to FIG. 4, the first housing 11 of the hand warmer 100a is formed by clasping a first front housing 111 and a first rear housing 112, creating an internal cavity to house the battery 2 and heating control circuit 3. The front side of the first front housing 111 and the rear side of the first rear housing 112 are each provided with an installation window 102 for mounting the heating element 4 and heat-conducting portion 4. Thus, the front and rear surfaces of the housing 1 each include a heat-conducting portion 5 to enable bidirectional heat transfer. Within the installation windows 102, the heat-conducting portion 5 is pressed against the heating element 4 and fixed via snap-fit connections to the first front housing 111 or first rear housing 112, ensuring tight contact for optimal heat transfer.

Referring to FIG. 4, the heating control circuit 3 includes a charging interface 7 and a power button 8. The charging interface 7 connects to an external power source to charge the battery 2 or powers external devices (e.g., smartphones). The power button 9 controls the hand warmer's on/off state.

Preferably, the heating element 4 in the embodiment is a flexible sheet material to conform to the installation windows 102 of the housing 1.

Further, the heating element 4 is a polyimide (PI) flexible heating film. This film uses a polyimide layer as the outer insulation and metal foil or wires as the internal conductive heating body, formed via high-temperature and high-pressure lamination. The PI flexible heating film not only adapts to curved installations but also exhibits excellent insulation and high heating efficiency.

In the embodiment, the second housing 12 is a cover attached to the upper ends of the first front housing 111 and first rear housing 112, compressing the hooking portion 61 of the soft silicone cover 6 to prevent loosening or detachment after installation.

Referring to FIG. 3, the pouch-like soft silicone cover 6 in the embodiment defines a receiving cavity 62 and an installation opening 63 connected to the receiving cavity 61. The first housing 11 of the hand warmer 100a is inserted into the receiving cavity 62 via the installation opening 63, enabling the soft silicone cover 6 to envelop the first housing 11 and the heat-conducting portions 5 on its front and rear sides.

In the embodiment, as illustrated in FIGS. 2 and 3, the soft silicone cover 6 is a pouch-like structure sealed at the front, rear, left, right, and bottom sides, with an open upper end serving as the installation opening 62. The hooking portion 61 is disposed on the inner walls at the front and rear sides of the installation opening 63. During assembly, the soft silicone cover 6 is sleeved upward from the bottom of the first housing 11, and the second housing 12 is then clasped onto the upper end of the first housing 11 to compression-fixed the hooking portion 61.

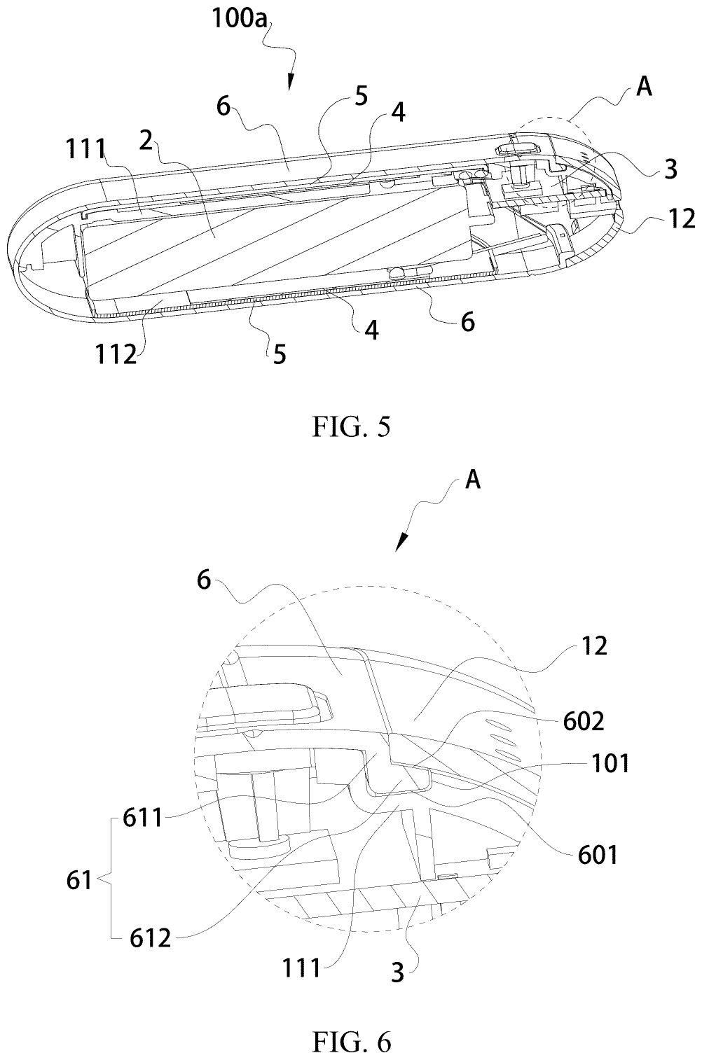

Specifically, referring to FIGS. 5 to 7, the soft silicone cover 6 extends outward at the installation opening 63 to form an L-shaped hooking portion 61, while the housing 1 is provided with a slot 101 to accommodate the L-shaped hooking portion 61. The hooking portion 61 is bent into an L-shaped structure. Correspondingly, both the first front housing 111 and first rear housing 112 are provided with slots 101 to receive the hooking portion 61. As shown in FIG. 6, the inner wall surface 601 of the hooking portion 61 engages with the slot 101 on the first front housing 111. Once the inner wall 601 is locked into the slot 101, the entire soft silicone cover 6 is tensioned to tightly adhere to the housing 1 and heat-conducting portion 2. Simultaneously, the outer wall surface 602 of the hooking portion 61 is compressed by the second housing 12, firmly securing the soft silicone cover 6 to the housing 1 to prevent loosening or detachment during use. In the embodiment, the soft silicone cover 6 is configured as a flat pouch-like structure. Accordingly, it can be understood that the hooking portions 61 are symmetrically provided on the left and right sections of the soft silicone cover 6. Correspondingly, the housing 1 is provided with slots 101 on both its front and rear surfaces. Specifically, each slot 101 is formed by a gap between the first housing 11 and the second housing 12 when mated. The slot 101 is a tortuous groove adapted to the hooking portion 61 to resist disengagement. It can be understood that the slot 101 has a cross-section in the shape of the letter L to accommodate the hooking portion 61.

In the embodiment, the L-shaped hooking portion 61 includes a first hooking arm 611 and a second hooking arm 612 connected perpendicularly. The first hooking arm 611 embeds into the slot 101, while the second hooking arm 612 extends toward the second housing 12, facilitating compression and fixation by the second housing 12.

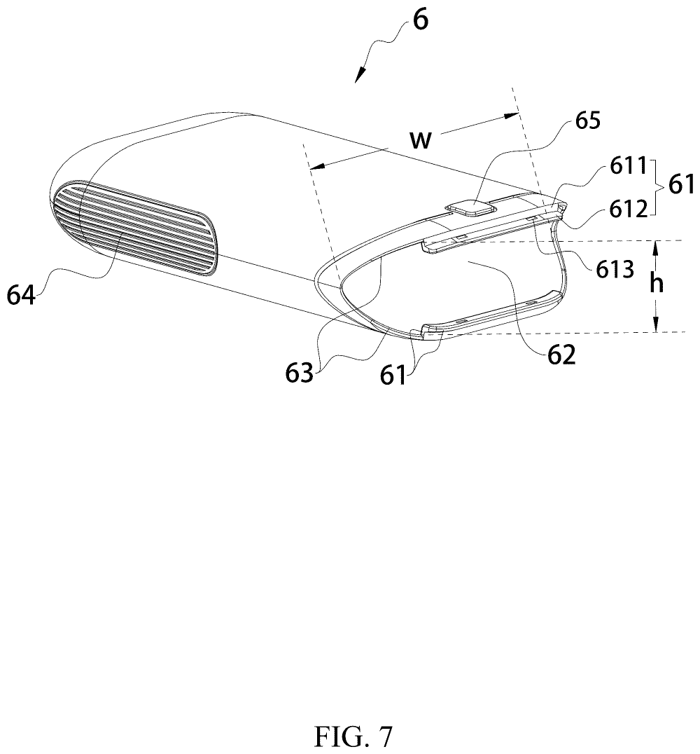

Preferably, as shown in FIG. 7, the height h and width w of the receiving cavity 62 in the soft silicone cover 6 are 0.1 mm to 0.13 mm smaller (optimally 0.1 mm) than the corresponding dimensions of the housing 1. This design ensures smooth insertion of the housing 1 into the soft silicone cover 6 while leveraging the cover's elastic deformation to tightly envelop the housing I and heat-conducting portion 5, achieving seamless contact for efficient heat transfer.

Preferably, referring to FIGS. 1 and 7, the outer surface of the soft silicone cover 6 is provided with anti-slip ribs 64 to enhance grip stability and prevent accidental drops.

The outer surface of the soft silicone cover 6 further includes a button 65, corresponding to a press block 103 on the housing 1. The inner surface of the press block 103 abuts against the switch 9 on the control circuit, while its outer surface is covered by the button 62. This design conceals the press block 103 for improved aesthetics.

Preferably, the press block 103 on the housing 1 is an integrally formed hollow structure with elastic reset functionality. After being pressed, the press block 103 automatically rebounds, enhancing operational feedback.

The thickness of the soft silicone cover 6 in the embodiment ranges from 0.8 mm to 1.2 mm (optimally 1.0 mm), balancing structural integrity, lightweight design, and tactile comfort.

Preferably, the housing 1 is provided with a visible LED indicator 8 electrically connected to the heating control circuit 3. The LED indicator 8 displays operational or charging/discharging statuses, improving user convenience.

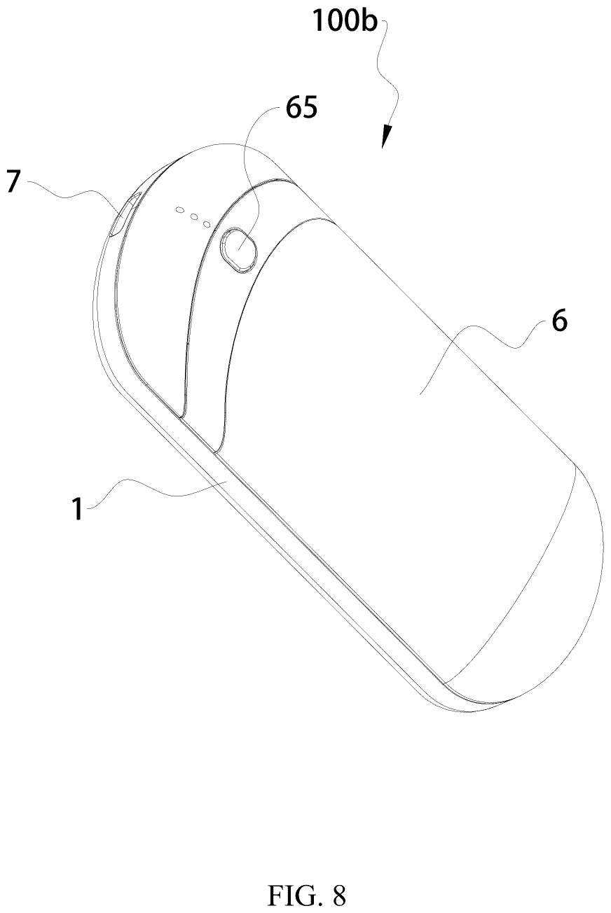

Referring to FIGS. 8 to 12, the present disclosure provides a second embodiment, a hand warmer 100b. Compared to the hand warmer 100a of the first embodiment, the primary distinction lies in the soft silicone cover 6, which adopts a sheet-like structure that fully covers the heat-conducting portion 5 without enveloping the entire housing 1. This design suits hand warmers with single-sided heating structures, such as those with a heat-conducting portion 5 only on the front or rear surface. In the embodiment, the hand warmer 100b includes the heat-conducting portion 5 solely on the front surface of the housing 1, with no rear-side thermal structure.

This configuration also supports combinatorial usage, two identical hand warmers 100b may be combined via magnetic attraction, plug-in, or snap-fit connections to form a dual-hand warmer set for left and right hands, offering flexible and convenient heating. Thus, the soft silicone cover 6 in the present disclosure is compatible with both standalone hand warmers and modular set applications.

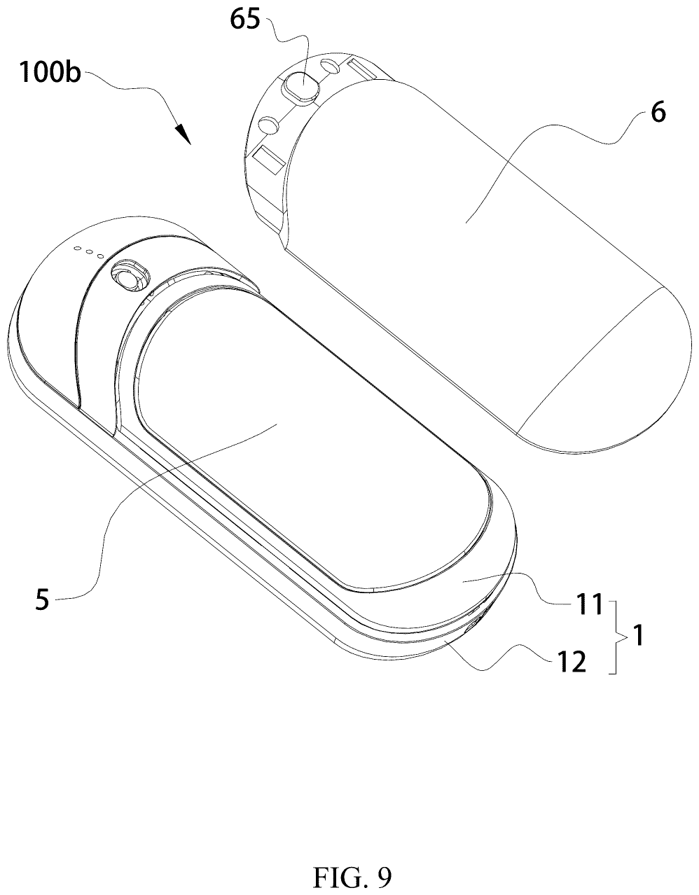

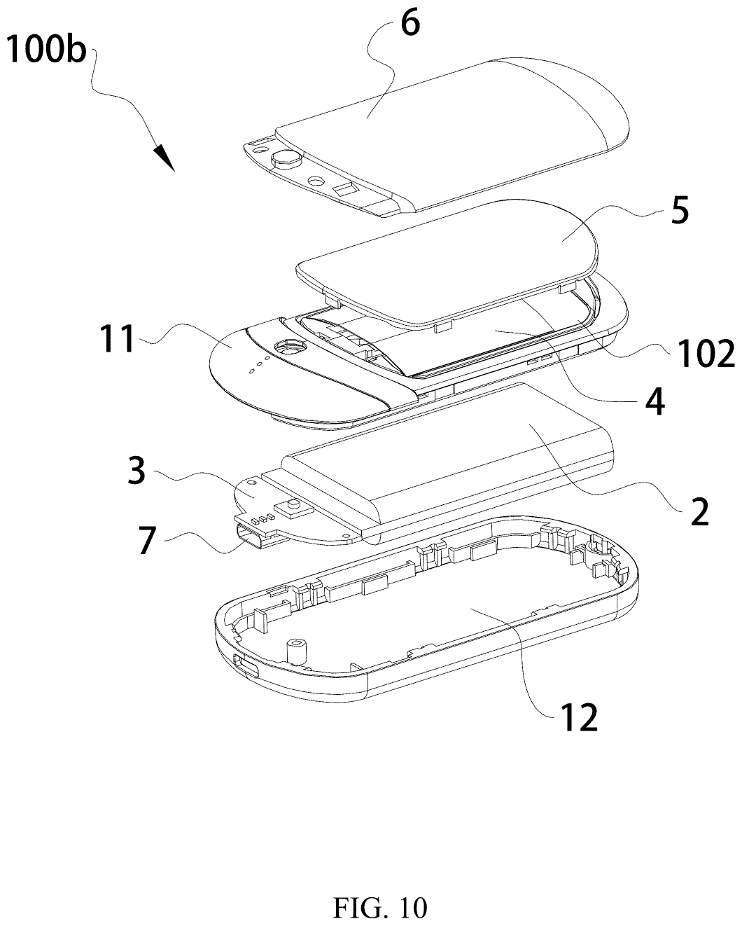

In addition, the second embodiment is further distinguished from the first embodiment as follows: referring to FIGS. 9 and 10, the first housing 11 serves as the front housing, the second housing 12 acts as the rear housing. The two components are clasped to form the housing 1, creating an internal cavity to accommodate the battery and heating control circuit.

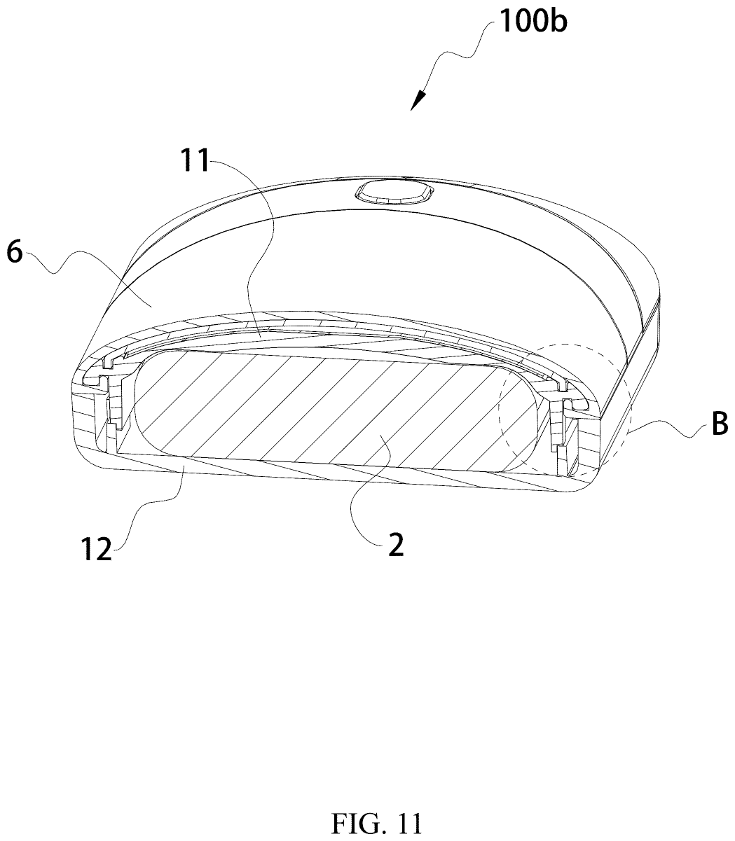

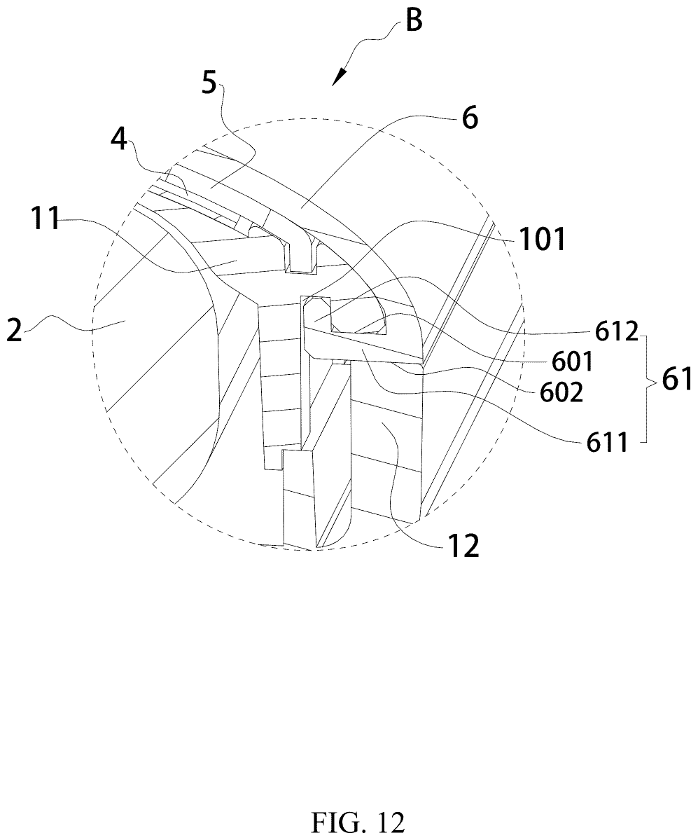

Referring to FIGS. 11 and 12, the sheet-like soft silicone cover 6 includes a peripherally arranged hooking portion 61. The hooking portion 61 is inwardly curved into an L-shaped structure (as shown in FIG. 12), where the second hooking arm 612 engages with the slot 101 on the first housing 11, and the first hooking arm 611 is compression-fixed by the second housing 12 for secure attachment.

Since the hand warmer 100b in the embodiment also incorporates the soft silicone cover 6 over the heat-conducting portion 5, it retains the advantages of the first embodiment, such as scratch protection, enhanced tactile comfort, and reduced heat dissipation, effectively extending operational duration.

While the present disclosure has been described with reference to a specific embodiment, the description of the disclosure is illustrative and is not to be construed as limiting the disclosure. Various of modifications to the present disclosure can be made to the exemplary embodiment by those skilled in the art without departing from the true spirit and scope of the disclosure as defined by the appended claims.

What is claimed is:

1. A hand warmer with silicone outer cover, comprising:
a housing, a battery, a heating control circuit electrically connected to the battery, and a heating element electrically connected to the heating control circuit, an outer surface of the housing is provided with a heat-conducting portion for transferring heat generated by the heating element, and wherein
a soft silicone cover is sleeved over the heat-conducting portion and at least a portion of the housing;
the soft silicone cover is configured as a flat pouch-like structure;
the soft silicone cover is provided with an outwardly extending hooking portion;
the housing is provided with a slot to accommodate the hooking portion to prevent detachment of the soft silicone cover from the housing.

2. The hand warmer with silicone outer cover according to claim 1, wherein the soft silicone cover is made of thermally conductive silicone material.

3. The hand warmer with silicone outer cover according to claim 1, wherein the housing includes a first housing and a second housing connected to each other, the hooking portion at an edge of the soft silicone cover is configured as an L-shaped hooking portion, an inner wall of the hooking portion clasps onto the first housing, an outer wall of the hooking portion is compression-fixed by the second housing.

4. The hand warmer with silicone outer cover according to claim 1, wherein the soft silicone cover is a sheet-like structure that fully covers the heat-conducting portion.

5. The hand warmer with silicone outer cover according to claim 1, wherein the soft silicone cover is a pouch-like structure enveloping the housing and the heat-conducting portion.

6. The hand warmer with silicone outer cover according to claim 5, wherein the soft silicone cover defines a receiving cavity, a height and a width of the receiving cavity are each 0.1 mm to 0.13 mm smaller than a height and a width of the housing.

7. The hand warmer with silicone outer cover according to claim 3, wherein the soft silicone cover is provided with an installation opening connected to the receiving cavity,
the L-shaped hooking portion is provided at the installation opening.

8. The hand warmer with silicone outer cover according to claim 7, wherein the slot is formed by a gap between the first housing and the second housing when mated.

9. The hand warmer with silicone outer cover according to claim 8, wherein the slot is configured as a tortuous groove to accommodate the L-shaped hooking portion and prevent disengagement thereof.

10. The hand warmer with silicone outer cover according to claim 8, wherein the L-shaped hooking portions are provided in at least two, a number of the slots corresponds to a number of the L-shaped hooking portions.

11. The hand warmer with silicone outer cover according to claim 1, wherein a thickness of the soft silicone cover ranges from 0.8 mm to 1.2 mm.

12. The hand warmer with silicone outer cover according to claim 1, wherein an outer surface of the soft silicone cover is provided with anti-slip ribs.

13. The hand warmer with silicone outer cover according to claim 3, wherein at least one of the first housing and the second housing is flat-shaped with a hollow cavity structure.

14. The hand warmer with silicone outer cover according to claim 3, wherein the hooking portion comprises a first hooking arm and a second hooking arm extending bendably from the first hooking arm, the first hooking arm is snapped into the slot, and the second hooking arm extends toward the second housing to facilitate being clamped by the second housing.
